(19) United States
(12) Patent Application Publication
(10) Pub. No.: US 2021/0401354 A1
(43) Pub. Date: Dec. 30, 2021

(54) BIOLOGICAL INFORMATION ACQUISITION DEVICE AND BIOLOGICAL INFORMATION ACQUISITION METHOD

(71) Applicant: OMRON HEALTHCARE Co., Ltd., Kyoto (JP)

(72) Inventors: Daisuke ISHIHARA, Kyoto (JP); Miki Okugawa, Kyoto (JP); Takehiro Hamaguchi, Kyoto (JP); Hisatomo Kowa, Hyogo (JP)

(21) Appl. No.: 17/447,503

(22) Filed: Sep. 13, 2021

Related U.S. Application Data
(63) Continuation of application No. PCT/JP2020/010438, filed on Mar. 11, 2020.

(30) Foreign Application Priority Data
Mar. 18, 2019 (JP) 2019-050507

(51) Int. Cl. A61B 5/00; A61B 5/021; A61B 5/024

(52) U.S. Cl. CPC A61B 5/4088; A61B 5/021; A61B 2503/08; A61B 5/0004; A61B 5/024; A61B 5/749

(57) ABSTRACT

A biological information acquisition device is provided that enables a cognitive function test to be received as part of routine healthcare management. Cognitive function test information is provided. Subsequently, biological information is acquired as an interference task. After acquiring the biological information, answers from the user to the previously provided cognitive function test information are checked.

### CROSS-REFERENCE TO RELATED APPLICATIONS

This application is the U.S. national stage application filed pursuant to 35 U.S.C. 365(c) and 120 as a continuation of International Patent Application No. PCT/JP2020/010438, filed Mar. 11, 2020, which application claims priority to Japanese Patent Application No. 2019-050507, filed Mar. 18, 2019, which applications are incorporated herein by reference in their entireties.

### TECHNICAL FIELD

The present invention relates to a biological information acquisition device and a biological information acquisition method.

### BACKGROUND ART

In recent years, due to an increasing number of elderly people, approach to dementia have been a challenge. Dementia refers to a condition in which intellectual functions temporarily normally developed are degraded persistently and a plurality of intellectual functions including a memory function are impaired, and thus social life is interfered. Impairment of the intellectual functions in dementia varies from a mild stage to a severe stage and is considered to advance in a step-wise fashion. Dementia advanced to a moderate stage interferes with daily life, but in a mild dementia state, lifestyle improvement and an anti-dementia drug may be effective, and thus early detection and early treatment are important.

As a cognitive function test for early detection of dementia, various tests have been proposed such as MMSE, Hasegawa Dementia Scale-Revised (HDS-R), WMS-R, ADAS-J, Cog, MoCA-J, and FCSRT. In a case of performing a diagnosis of dementia in a medical institution, a plurality of the cognitive function tests of these are performed in combination, and neurological consultation, MRI, and the like are also performed. Such diagnosis of dementia require a long time, which poses a hurdle for dementia test subjects such as elderly people to receive medical consultation, and this contributes to preventing early detection.

Patent Document 1 proposes a dementia diagnosis assistance system that provides a simplified diagnostic test for dementia in a short time during a waiting time in a medical institution.

### CITATION LIST

### Patent Literature

Patent Document 1: JP 2017-217051 A

### SUMMARY OF INVENTION

### Technical Problem

However, in order to take such a simplified diagnostic test, a test subject needs to visit a medical institution where the system is installed. Thus, the test is prevented from being readily taken as part of routine healthcare management.

In view of the known technology as described above, an object of the present invention is to provide a biological information acquisition device that enables the cognitive function test to be received as part of routine healthcare management.

### Solution to Problem

In order to achieve the object described above, the present invention provides a biological information acquisition device including a cognitive function test function, the biological information acquisition device including a test information provision unit configured to perform test information provision processing that provides a user with test information for testing cognitive function, biological information acquisition unit configured to perform biological information acquisition processing that acquires biological information of the user, and answer check unit configured to perform answer check processing that checks answers from the user to the test information, wherein the test information provision processing, the acquisition of the biological information of the user, and the check of the answers from the user to the test information are performed in this order.

This allows the cognitive function test to be received when biological information is acquired as part of routine healthcare management. In this regard, the biological information includes, but is not limited to, blood pressure information such as systolic blood pressure, diastolic blood pressure, and pulse, body composition such as body weight and body fat percentage, exercise amount such as the number of steps and calorie consumption, heart rate, electrocardiographic data, respiratory rate, body temperature, and the like.

Additionally, as a cognitive function test, an appropriate cognitive function test such as a test dedicated to memorization can be employed with reference to various tests such as MMSE, Hasegawa Dementia Scale-Revised (HDS-R), WMS-R, ADAS-J, Cog, MoCA-J, and FCSRT, which are well known.

In addition, in the present invention, an amount of time for the biological information acquisition processing may be matched with an amount of time that needs to elapse after end of the test information provision processing until initiation of the answer check processing.

In this way, by adapting the amount of time for the biological information acquisition processing to the amount of time to elapse which is required after the end of the test information provision processing until the initiation of the answer check processing, the biological information acquisition processing can be effectively utilized for the cognitive function test.

Additionally, in the present invention, contents of the test information may be changed such that the biological information acquisition processing is performed during the amount of time to elapse after the end of the test information provision processing until the initiation of the answer check processing.

In this regard, the contents of the test information include, but are not limited to, the type of the test such as image memorization and word memorization, difficulty level, number of problems, and the like.

Additionally, in the present invention, a length of time for performing the biological information acquisition processing may be determined such that the biological information acquisition processing is performed during an amount of time that needs to elapse after the end of the test information provision processing until the initiation of the answer check processing.

In addition, in the present invention, the biological information acquisition processing may be processing that acquires the biological information by measurement.

This allows the cognitive function test to be received when the biological information is measured as part of routine healthcare management.

Additionally, in the present invention, the biological information acquisition processing may be acquisition processing that receives transfer of the biological information measured by a biological information measurement instrument.

This allows the cognitive function test to be received by utilizing the opportunity to transfer the biological information measured as part of routine healthcare management. In addition, the transfer received need not be limited to the biological information measured by the biological information measurement instrument but may include related information associated with the biological information and including environment information such as air temperature and atmospheric pressure.

Additionally, the present invention provides a biological information acquisition method including a cognitive function test function, the biological information acquisition method including (a) providing a user with test information for testing cognitive function, (b) acquiring biological information of the user, and (c) checking answers from the user to the test information, with (a), (b) and (c) being performed in this order.

This allows the cognitive function test to be received when biological information is acquired as part of routine healthcare management.

In this regard, the biological information includes, but is not limited to, blood pressure information such as systolic blood pressure, diastolic blood pressure, and pulse, body composition such as body weight and body fat percentage, exercise amount such as the number of steps and calorie consumption, heart rate, electrocardiographic data, respiratory rate, body temperature, and the like. Additionally, as a cognitive function test, an appropriate cognitive function test such as a test dedicated to memorization can be employed with reference to various tests such as MMSE, Hasegawa Dementia Scale-Revised (HDS-R), WMS-R, ADAS-J, Cog, MoCA-J, and FCSRT, which are well known.

### Advantageous Effects of Invention

In accordance with the present invention, a biological information acquisition device can be provided that enables the cognitive function test to be received as part of routine healthcare management.

### BRIEF DESCRIPTION OF DRAWINGS

FIG. 1 is a block diagram illustrating an overview of a configuration example of a biological information measurement system according to a first embodiment.

FIG. 2 is a flowchart illustrating a processing procedure for a blood pressure information measurement method of a biological information measurement system according to the first embodiment.

FIG. 3 is a flowchart illustrating a processing procedure for provision of test information in the biological information measurement system according to the first embodiment.

FIG. 4 is a diagram illustrating a display example of a touch panel display of the biological information measurement system according to the first embodiment.

FIG. 5 is a diagram illustrating transition of words displayed in provision of test information in the biological information measurement system according to the first embodiment.

FIG. 6 is a flowchart illustrating a procedure for blood pressure information measurement processing using a blood pressure monitor of the biological information measurement system according to the first embodiment.

FIG. 7 is a diagram illustrating a display example of a touch panel display in the blood pressure information measurement processing using the blood pressure monitor of the biological information measurement system according to the first embodiment.

FIG. 8 is a flowchart illustrating a processing procedure for answer check in the biological information measurement system according to the first embodiment.

FIG. 9 is a diagram illustrating transition of words displayed in provision of test information in a biological information measurement system according to a second embodiment.

FIG. 10 is a flowchart illustrating a processing procedure for answer check in the biological information measurement system according to the second embodiment.

FIG. 11 is a diagram illustrating transition of words provided in answer check processing of the biological information measurement system according to the second embodiment.

FIG. 12 is a flowchart illustrating a processing procedure for provision of test information in a biological information measurement system according to a third embodiment.

FIG. 13 is a diagram illustrating a display example of a touch panel display in the test information provision processing according to the third embodiment.

FIG. 14 is a diagram illustrating transition of illustrations provided in the test information provision processing according to the third embodiment.

FIG. 15 is a flowchart illustrating a processing procedure for answer check in the biological information measurement system according to the third embodiment.

FIG. 16 is a flowchart illustrating a processing procedure for provision of test information in a biological information measurement system according to a fourth embodiment.

FIG. 17 is a flowchart illustrating a processing procedure for reception of transferred biological information in the biological information measurement system according to the fourth embodiment.

### DESCRIPTION OF EMBODIMENTS

Specific embodiments of the present invention will be described below with reference to the drawings.

### First Embodiment

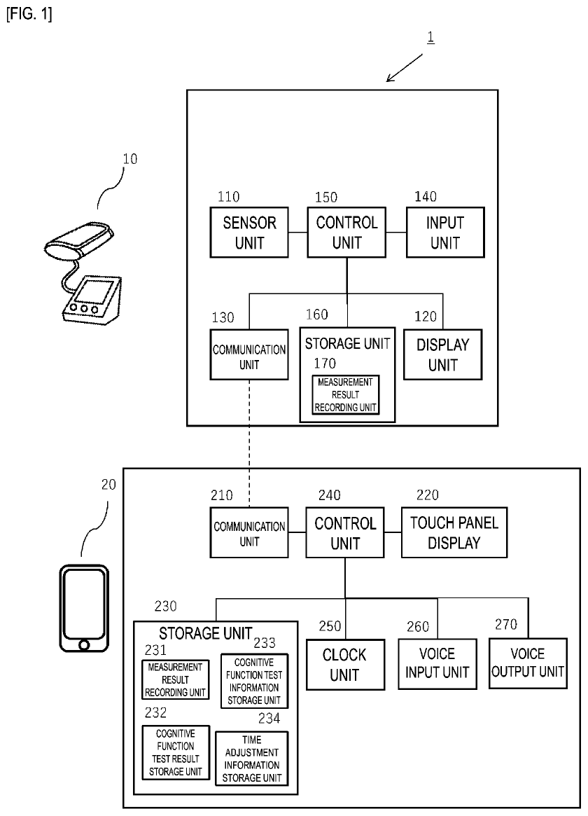

First, an example of the embodiments of the present invention will be described with reference to FIG. 1 to FIG. 15. However, the dimensions, materials, shapes, relative arrangements, and other characteristics of the components described in this embodiment are not intended to limit the scope of this invention unless otherwise indicated.

System Configuration

FIG. 1 is a schematic diagram illustrating a configuration example of a biological information measurement system 1 used as a biological information acquisition device according to the present embodiment. As illustrated in FIG. 1, the biological information measurement system 1 includes a blood pressure monitor 10 used as a biological information measurement instrument, and a smartphone 20 used as a terminal. The blood pressure monitor 10 and the smartphone 20 are configured to be communicable with each other. A communication method is not particularly limited, and the communication method employed may include a wireless communication method such as Bluetooth (trade name), infrared communication, or information transmission using ultrasonic waves, or a wired communication method with connection via a cable, a connector, or the like.

Blood Pressure Monitor

The blood pressure monitor 10 of the present embodiment is a measurement apparatus that measures a blood pressure of a user using a so-called oscillometric method and, as illustrated in FIG. 1, includes a sensor unit 110, a display unit 120, a communication unit 130, an input unit 140, a control unit 150, and a storage unit 160.

The sensor unit 110 includes a pressure sensor disposed in a cuff portion of the blood pressure monitor 10, and detects a pulse wave from a blood vessel of the user under a proper cuff pressure. The blood pressure monitor 10 of the present embodiment is capable of measuring a pulse in addition to a systolic blood pressure and a diastolic blood pressure on the basis of the pulse wave detected by the sensor unit. The values of the systolic blood pressure, the diastolic blood pressure, and the pulse are collectively referred to as blood pressure information. In the present embodiment, the blood pressure information corresponds to the biological information.

The display unit 120 is formed by, for example, a liquid crystal display, and displays the calculated blood pressure information.

The communication unit 130 is a communication interface configured to transmit and receive signals to and from the smartphone 20. In a case of using the wireless communication method, the communication unit 130 is a communication antenna that transmits and receives radio waves including radio waves for short-range wireless communication. However, the communication unit 130 is not limited to the communication antenna, and a desired known technology can be employed. Additionally, the communication unit 130 may use the wired communication method, and a desired known technology can be employed. The communication unit 130 transmits information including an identification number (for example, a serial number) that is respectively unique to the blood pressure monitor, and measured blood pressure information.

The input unit 140 is an input unit such as a button or a touch panel display that receives input from the user, and receives various operations from the user, such as power-on and -off, initiation of measurement, and selection of an item.

The control unit 150 is a unit for controlling the blood pressure monitor 10, and includes a Central Processing Unit (CPU), for example. In response to reception of an instruction to initiate measurement, from the user via the input unit 140, the control unit 150 pressurizes the cuff and, under an appropriate cuff pressure, calculates the blood pressure information based on the pulse wave detected by the sensor unit 110. Then, the calculated value is displayed on the display unit 120. In addition, each component of the blood pressure monitor 10 is controlled to execute processing corresponding to the operation of the user via the input unit 140.

A storage unit 160 includes a main storage device such as a random access memory (RAM) and, for example, a long-term storage medium such as a flash memory, and stores various types of information such as application programs and the blood pressure information.

A measurement result recording unit 170 is a non-volatile memory included in the storage unit 160, and records information such as blood pressure information measured by the sensor unit 110.

Smartphone

As illustrated in FIG. 1, the smartphone 20 includes a communication unit 210, a touch panel display 220, a storage unit 230, a control unit 240, a clock unit 250, a voice input unit 260, and a voice output unit 270.

The communication unit 210 is a communication interface configured to transmit and receive signals to and from the blood pressure monitor 10. In a case of using the wireless communication method, the communication unit 210 is a communication antenna that transmits and receives radio waves including radio waves for short-range wireless communication. In addition to receiving measurement information transmitted from the blood pressure monitor 10, the communication unit 210 also transmits and receives radio waves to and from other electronic apparatuses or base stations. The communication unit 210 may use the wired communication method, and a desired known technology can be employed.

The touch panel display 220 is used both as a display unit and as an input unit. The touch panel display 220 displays various pieces of information to the user, and receives various inputs from the user.

The storage unit 230 includes, for example, a long-term storage medium such as flash memory, in addition to a main storage device such as a RAM, and stores various types of information such as application programs and the biological information.

A measurement result recording unit 231 is a non-volatile memory included in the storage unit 230, and information recorded in the measurement result recording unit 231 includes blood pressure information received from the blood pressure monitor 10 and information such as measurement date and time at which the blood pressure information is measured.

A cognitive function test result recording unit 232 is a non-volatile memory included in the storage unit 230, and the results of the cognitive function test described below are recorded in the cognitive function test result recording unit 232. The results of the cognitive function test include, but are not limited to, information of the dates and times at which the cognitive function tests are performed, the contents of the test, and evaluation information such as the correct answer rate.

A cognitive function test information storage unit 233 is a non-volatile memory included in the storage unit 230, and stores data such as words and illustrations used in the cognitive function test and programs for conducting the cognitive function test. Additionally, in accordance with the contents of the cognitive function test, information is also stored that is related to the amount of time that needs to elapse, which is required after provision of test information until check of answers from the user to the test information.

A time adjustment information storage unit 234 is a non-volatile memory included in the storage unit 230. The time adjustment information storage unit 234 stores time adjustment information used to adjust blood pressure measurement time and the amount of time after the execution of the cognitive function test until the check of the answers from the user. The time adjustment information is information provided to the user through the touch panel display 220 and the voice output unit 270, and in order to provide in accordance with time to be adjusted, various types of information, such as visual effects for display of measurement values, music, moving images, programs for conversations with characters are stored.

The control unit 240 is an unit for controlling a smart phone, and is configured to include, for example, a CPU, and executes various programs stored in the storage unit 230, thereby exhibiting the functions corresponding thereto.

The clock unit 250 has a clocking function and holds current date and time information.

The voice input unit 260 includes a microphone that converts voice into an electrical signal, and receives input of voice information.

The voice output unit 270 includes a speaker that converts an electrical signal into voice, and outputs voice information.

Blood Pressure Information Acquisition Method

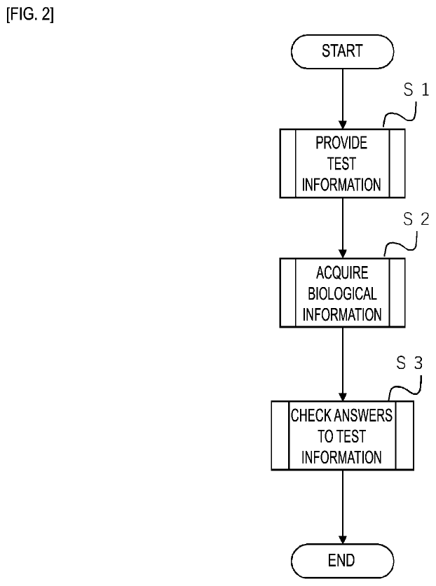

FIG. 2 is a flowchart illustrating a processing procedure for a biological information acquisition method including cognitive function test processing, biological information acquisition processing, and answer check processing. In this regard, blood pressure information used as biological information is acquired by measurement. In other words, the biological information acquisition processing is implemented as a blood pressure information measurement processing.

First, information for the cognitive function test is provided to the user via the smartphone 20 (step S1).

Then, blood pressure information, used as biological information, is measured by the blood pressure monitor 10 (step S2). Then, answers to information for the cognitive function test are received from the user and checked, via the smartphone 20 (step S3).

Cognitive Function Test Information Provision Processing

As a cognitive function test, various tests have been proposed such as MMSE, Hasegawa Dementia Scale-Revised (HDS-R), WMS-R, ADAS-J, Cog, MoCA-J, and FCSRT. In the present embodiment, in order to effectively utilize the amount of time during which the blood pressure information is measured using the blood pressure monitor 10, a test dedicated to memorization is conducted, but the present invention is not limited to such a test. When the cognitive function test is conducted, in a case where a predetermined amount of time is required to elapse before the user answers or a predetermined interference problem is required, the user can be prompted to measure the blood pressure information or to perform tasks including the measurement as an alternative to the elapse of the predetermined amount of time or the interference problem. This allows the cognitive function test to be readily conducted in conjunction with the measurement of the blood pressure information that is routine healthcare management. This in turn enables easing of uncomfortable feeling in taking the cognitive function test and also enables a reduction in time and effort required to take the cognitive function test, allowing early detection of dementia to be expected.

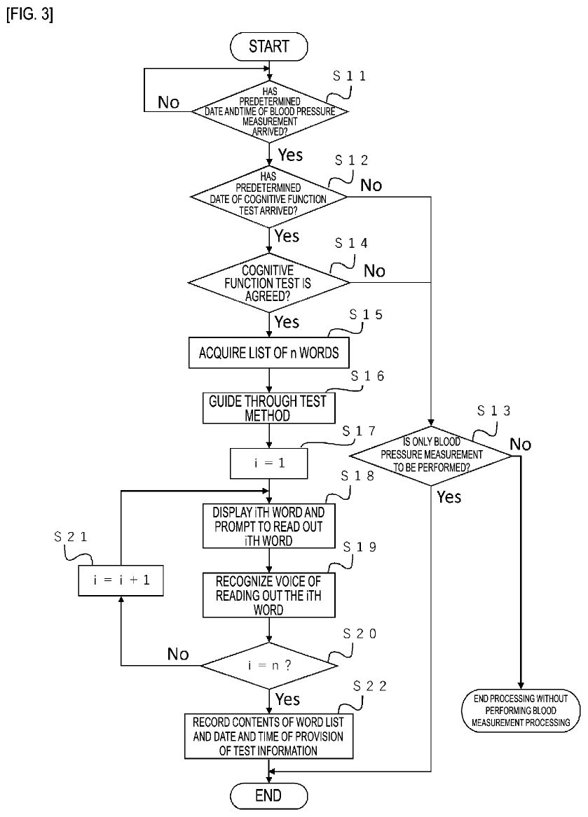

FIG. 3 is a flowchart illustrating a processing procedure for provision of cognitive function test information. The cognitive function test of the present embodiment is referred to as word recalling, in which the user is prompted to memorize a plurality of displayed words and to orally speak the previously displayed words after a predetermined amount of time or after a predetermined interference task. The cognitive function test information provision processing described below, including other examples, is implemented by the control unit 240 of the smartphone 20 by executing a program stored in the storage unit 230. In other words, a test information provision unit is configured by including the touch panel display 220, the storage unit 230, the clock unit 250, the voice input unit 260, and the voice output unit 270, and the control unit 240 cooperating the units 220, 230, 250, 260, and 270. Additionally, the cognitive function test functions are implemented by executing the cognitive function test information provision processing and oral check processing described below.

When the date and time preset by the user as the date and time of blood pressure information measurement arrives (step S11), whether the date preset as the date of the cognitive function test has arrived (step S12). In a case where in step S12, the date of the cognitive function test is determined not to have arrived, the user is prompted via the touch panel display 220 to determine whether to perform only blood pressure measurement (step S13). In step S13, in a case where an input indicating that only blood pressure measurement is to be performed is received from the user via the touch panel display 220 or via the voice input unit 260, the cognitive function test information provision processing is ended and blood pressure measurement processing described below is performed. In this case, when the blood pressure measurement process ends, the processing is ended without execution of the answer check processing. In step S13, in a case where an input indicating that only blood pressure measurement is not to be performed is received from the user via the touch panel display 220 or via the voice input unit 260, the processing is ended with neither the blood pressure measurement processing nor the answer check processing executed.

In a case where it is determined in step S12 that the date of the cognitive function test is determined to have arrived, then a message “Would you like to take the cognitive level test?” is displayed on the touch panel display of the smartphone 20, or the voice output unit 270 outputs the voice of the corresponding message and asks the user for agreement (step S14).

At this time, a “yes” and a “no” buttons are displayed on the touch panel display 220 of the smartphone 20, and whether the user has touched one of the buttons is detected, and whether the user has agreed to take the test is determined. Alternatively, determination may be performed by receiving an oral answer of the user from the voice input unit 260 of the smartphone 20.

In a case where the user does not agree, then the processing proceeds to step S13.

In a case where the user agrees, a list of n words stored in the cognitive function test information storage unit 233 of the storage unit 230 is acquired (step S15). The cognitive function test information storage unit 233 may store a word list, or may be configured to extract n words from a group of words to constitute a word list. At this time, the words constituting the group of words may be stored along with categories to which the respective words belong.

Then, a cognitive function test method to be performed is displayed on the touch panel display 220 of the smartphone 20 to guide through the method (step S16). Here, a message guiding through the method of the cognitive function test is displayed, such as “Now, n words are displayed one by one. Please read out and memorize the displayed words.” The message may also be output by the voice output unit 270 of the smartphone 20.

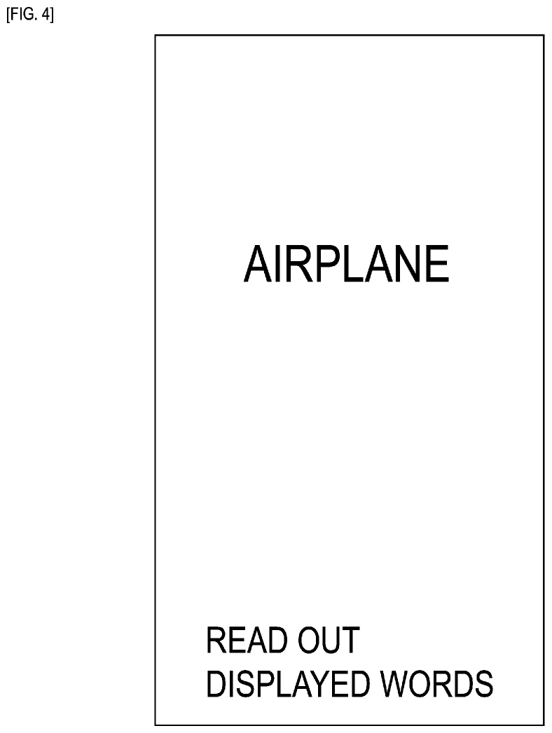

Next, i=1 is set (step S17). As illustrated in FIG. 4, the first word of the word list acquired in step S13 is displayed on the touch panel display 220, and a message prompting the user to read out the displayed word is displayed (step S18). In this case, the word “airplane” is displayed on the touch panel display 220. The message prompting to read out the displayed word may be output via the voice output unit 270.

From the voice acquired via the voice input unit 260 of the smartphone 20, the voice of the user reading out the ith word is recognized (step S19). In a case where the voice of the user reading out the ith word fails to be recognized within a predetermined amount of time, a message prompting the user again to read out the displayed word may be displayed on the touch panel display 220 or output via the voice output unit 270. Additionally, in a case where the voice of the user reading out the ith word fails to be recognized within the predetermined amount of time, the processing may proceed to step S20.

When the voice of the user reading out the ith word is recognized, whether i=n is determined (step S20).

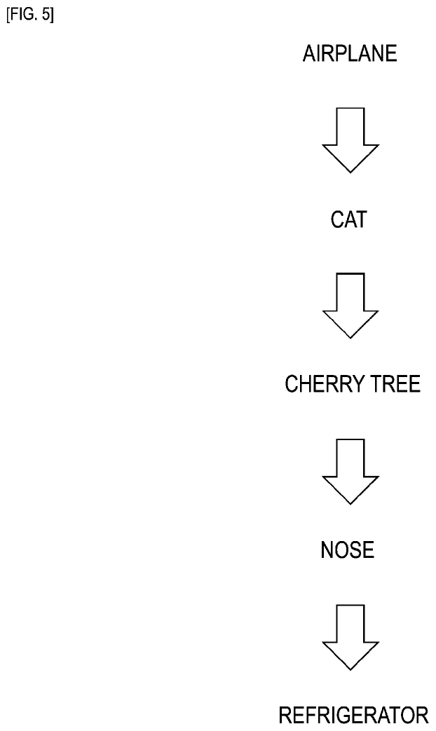

In a case where i=n does not hold true at step S20, then i=i+1 is set (step S21), and the processing returns to step S18 to repeat the processing from step S18 onward for the next word in the word list acquired in step S15. In this way, as illustrated in FIG. 5, n different words are sequentially displayed on the touch panel display 220 of the smartphone 20. In this case, the word list is constituted by five words of an airplane, a cat, a cherry tree, a nose, and a refrigerator. The words displayed at this time all belong to different categories, such as a vehicle, an animal, a flower, a part of a body, a home electrical appliance, and the like. The number n of words displayed is not limited to 5, and may be set as appropriate. In a case where similar tests are repeated a plurality of times, the number of displayed words increases each time, for example, five words on the first test and seven words on the second test.

In a case where i=n holds true at step S20, the contents of the word list acquired in step S15 and the date and time of provision of test information are stored in the cognitive function test result recording unit 232 of the storage unit 230 (step S22). In this case, the date and time of provision of the test information is, for example, the date and time of recognition of the voice of reading out the nth word. Then, the provision processing for the cognitive function test information is ended.

When the provision processing for the cognitive function test information ends as described above, the processing proceeds to the blood pressure information measurement processing (step S2), as illustrated in FIG. 2.

Blood Pressure Information Measurement Processing

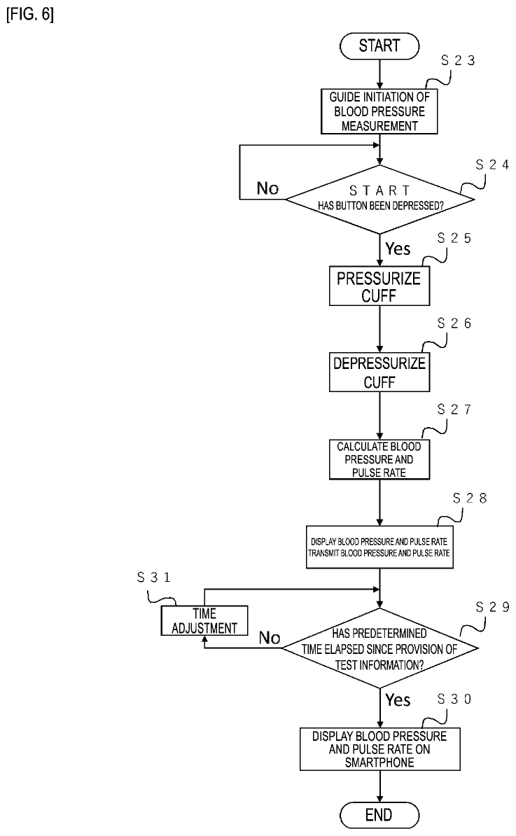

FIG. 6 is a flowchart illustrating a processing procedure for measurement of blood pressure information. The blood pressure information measurement processing described below, including other examples, is implemented by the control unit 240 of the smartphone 20 by executing a program stored in the storage unit 230 and cooperating with the blood pressure monitor 10. At this time, in the blood pressure monitor 10, the control unit 150 similarly executes a program stored in the storage unit 160. The biological information measuring unit used as a biological information acquisition unit includes the touch panel display 220, the storage unit 230, the clock unit 250, the voice input unit 260 and the voice output unit 270, the communication unit 210, and the control unit 240 that are belonging to the smartphone 20, in which the control unit 240 cooperates with the units 220, 230, 250, 260, 270, 210, and 240. The biological information measuring unit further includes at least the sensor unit 110, the storage unit 160, the communication unit 130, and the control unit 150 that are belonging to the blood pressure monitor 10, in which the control unit 150 cooperates with the units 110, 160, and 130.

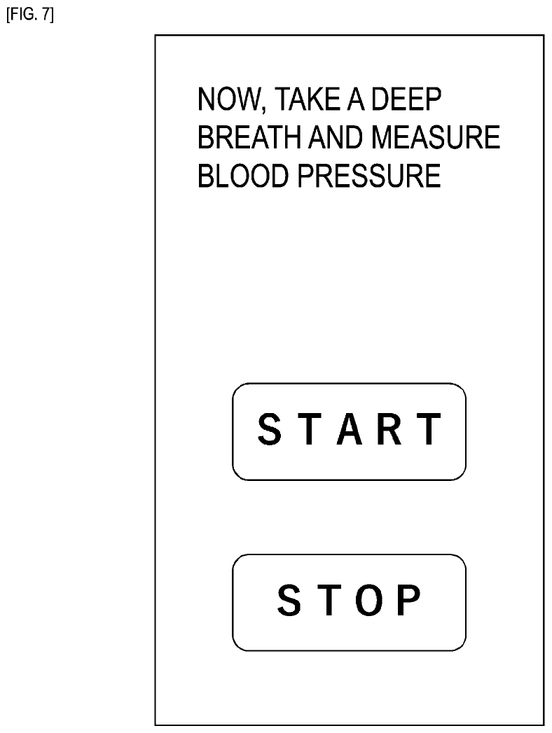

First, the user is guided to the initiation of the blood pressure measurement (step S23). At this time, as illustrated in FIG. 7, a “START” and a “STOP” buttons are displayed on the touch panel display 220 of the smartphone 20, and a message prompting blood pressure information measurement, as “Now, take a deep breath and measure the blood pressure.” is displayed on the touch panel display 220 or output from the voice output unit 270. In this regard, a message describing an operation for measuring the blood pressure may be displayed on the touch panel display 220 or output from the voice output unit 270; a specific example of the message instructs to wrap a cuff around the arm. At this time, predetermined initialization of the blood pressure monitor 10 is performed.

The smartphone 20 waits until the user wraps the cuff around the arm and depresses the “START” button displayed on the touch panel display 220 of the smartphone 20 or the user inputs the voice “start” via the voice input unit 260 (step 24).

When the “start” button is depressed or the voice “start” is detected, the smartphone 20 transmits, to the blood pressure monitor 10, an instruction to initiate blood pressure measurement, and in response to reception of the instruction, the blood pressure monitor 10 pressurizes the cuff pressure (step S25). When the cuff is pressurized to a predetermined pressure, the cuff pressure is gradually reduced (step S26). Then, a known method is used to calculate the systolic blood pressure, the diastolic blood pressure, and the pulse rate (step S27).

When the systolic blood pressure, the diastolic blood pressure, and the pulse rate are calculated in the blood pressure monitor 10, the systolic blood pressure, diastolic blood pressure, and pulse rate calculated are displayed on the display unit 120 of the blood pressure monitor 10, and the blood pressure information including the systolic blood pressure is transmitted to the smartphone 20 (step S28).

In response to reception of blood pressure information from the blood pressure monitor 10, the smartphone 20 recognizes that the measurement of blood pressure information by the blood pressure monitor 10 has ended. Then, the smartphone 20 determines whether a predetermined amount of time has elapsed since the date and time of provision of the cognitive function test information stored in step S22 (step S29). The predetermined amount of time is the amount of time to be unoccupied from provision of the cognitive function test information, such as display of words, until the check of answers such as recalling of words by the user, in accordance with the contents of the cognitive function test performed in step S1. Such a predetermined time is stored in the cognitive function test information storage unit 233 of the storage unit 230.

In step S28, in a case where the determination is made that the predetermined amount of time has elapsed since the date and time of provision of cognitive function test information, the systolic blood pressure, diastolic blood pressure, and pulse rate calculated in step S27 are displayed on the touch panel display 220 of the smartphone 20 (step S30), and the processing is ended. In step S29, the calculated systolic blood pressure, diastolic blood pressure, and pulse rate may be output via the voice output unit 270.

In step S29, in a case where the determination is made that the predetermined amount of time has not elapsed since the date and time of provision of cognitive function test information, the processing proceeds to step S31. In step S31, time adjustment processing is performed to adjust the time elapsing from the date and time of provision of cognitive function test information. In this regard, as an example of the time adjustment processing, time adjustment information stored in the time adjustment information storage unit 234 of the storage unit 230 is provided to the user via the touch panel display 220 and the voice output unit 270. In this regard, the difference between the amount of time to be unoccupied (time to elapse) from the provision of cognitive function test information until the check of answers and the current elapsed time is a time to be adjusted. The contents of the time adjustment information is selected according to such a time to be adjusted. The time of the blood pressure information measurement processing is determined including the time to provide the selected time adjustment information. The time adjustment information is, for example, music, moving images, games, conversations with characters, and the like, and these files and programs are read from the time adjustment information storage unit 234 and reproduced or executed to provide the time adjustment information to the user. These pieces of time adjustment information are provided, and the elapse of a predetermined amount of time is waited for since the date and time of provision of the cognitive function test information. In accordance with the difference described above, a plurality of types of information may be provided, or the provision may be stopped partway through one piece of information. The time adjustment information is not limited to the time adjustment information stored in the time adjustment information storage unit 234, and may be provided to the user by a method such as streaming from an external server via a network.

In this case, the time adjustment information is provided before display of the blood pressure information on the touch panel display 220 (step S30). The blood pressure information is already displayed on the display unit 120 of the blood pressure monitor 10 in step S28, and thus the current processing for displaying the blood pressure information may be simply omitted. However, by displaying, on a graph, a history including previous blood pressure information recorded in the measurement result recording unit 231 of the storage unit 230, along with display of the blood pressure information such as the systolic blood pressure acquired by the current measurement, or applying visual or auditory effects to the display of the current blood pressure information, or the like, the time adjustment information may be provided including information related to the current blood pressure information. Additionally, for provision of the time adjustment information, a message prompting the user to read out the display such as the systolic blood pressure may be displayed on the touch panel display 220 or output from the voice output unit 270. In addition, in a case where the time to be adjusted is short, for example, approximately one second, the display of blood pressure information (step S30) may be delayed slightly for the time adjustment processing.

Answer Check Processing

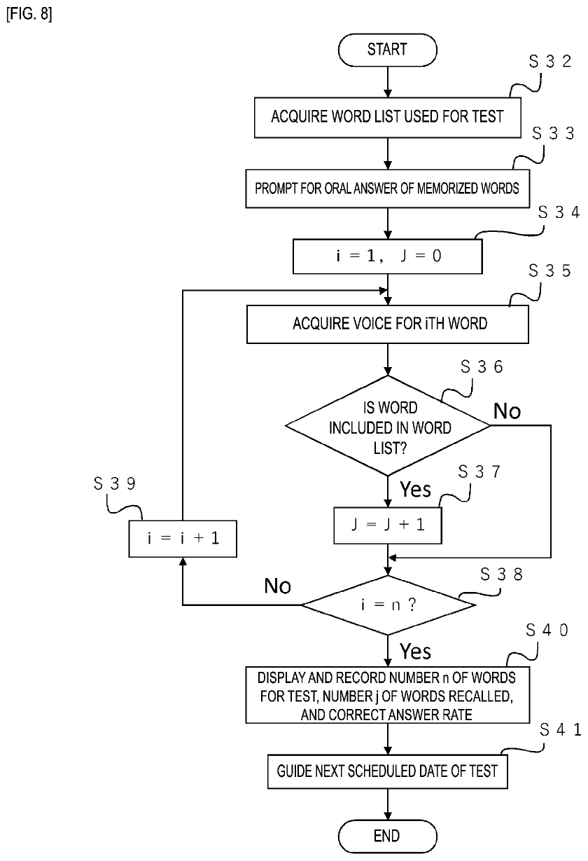

FIG. 8 is a flowchart illustrating a processing procedure for checking answers from the user to the cognitive function test information. The answer check processing described below, including other examples, is implemented by the control unit 240 of the smartphone 20 by executing a program stored in the storage unit 230. In other words, an answer check unit is constituted by the touch panel display 220, the storage unit 230, the clock unit 250, the voice input unit 260 and the voice output unit 270, and the control unit 240 cooperating with the units 220, 230, 250, 260, and 270.

In step S32, the word list used for the test is acquired from the cognitive function test result recording unit 232 of the storage unit 230.

Then, a message prompting the user to orally answer memorized words is displayed on the touch panel display 220 (step S33). For example, a message such as “Please speak memorized words” is displayed on the touch panel display 220. A message prompting the oral answer of the memorized words may be output via the voice output unit 270, and display and voice output may be combined.

Then, i=1 and j=0 are set (step S34). Then, the voice of the user for the ith word is acquired via the voice input unit 260 (step S35).

The voice acquired is analyzed to determine whether the word answered by the user is included in the word list acquired in step S32 (step S36).

In step S36, in a case where the word is determined to be included in the word list, j=j+1 is set (step S37) and the processing proceeds to step S38. In step S36, in a case where the word is determined not to be included in the word list, then the processing proceeds to step S38.

In step S38, whether i=n is determined.

In step S38, in a case where i=n does not hold true, then i=i+1 is set (step S39) and the processing returns to step S35 to wait for acquisition of the voice for the i+1th word.

In step S38, in a case where i=n holds true, the number n of words used in the current cognitive function test, the number j of words successfully recalled by the user, and the correct answer rate j/n are displayed on the touch panel display 220, and are recorded in the cognitive function test result recording unit 232 of the storage unit 230 (step S40).

Then, the user is guided to the next scheduled date of the cognitive function test (step S41). For example, a message is displayed on the touch panel display 220, such as “Thank you for your work, the test is over now. You have done very well. Please take the test three more times to produce final results. The next test will be conducted on Xth day.” The message may be output via the voice output unit 270.

The next scheduled date of the test is recorded in the cognitive function test result recording unit 232 of the storage unit 230, and when the blood pressure information on the day, the next cognitive function tests are prompted. Thus, accuracy of evaluation is increased by conducting the cognitive function test a plurality of times.

Thus, according to the present embodiment, the opportunity for the measurement of the blood pressure information performed as part of routine healthcare management is utilized to perform the cognitive function test. This enables lowering the hurdle of taking the cognitive function test, and allows early detection of dementia to be expected.

As described above, in the present embodiment, the blood pressure measurement by the blood pressure monitor 10 is initiated by transmitting, from the smartphone 20 to the blood pressure monitor 10, an instruction to initiate blood pressure measurement. However, the operations of the smartphone 20 and blood pressure monitor 10 performed to initiate blood pressure measurement are not limited to the operations described above. For example, in the smartphone 20, the user may be guided through the operation of the blood pressure monitor 10 by display on the touch panel display 220 or voice output from the voice output unit 270, and blood pressure measurement may be initiated by the user by depressing a button constituting the input unit 140 of the blood pressure monitor 10. In this case as well, the smartphone 20 receives, in step S28, the blood pressure information transmitted from the blood pressure monitor 10 to allow the end of the blood pressure measurement to be recognized. This enables subsequent processing to be similarly performed.

### Second Embodiment

A second embodiment of the present invention will be described below. The same reference numerals are used for components that are the same as those of the first embodiment, and detailed descriptions thereof are omitted.

The present embodiment is the same as the first embodiment in the configuration of the biological information system, the overall flow from the provision of the cognitive function test information through the measurement of the blood pressure information to the check of answers to the test information, and the contents of the blood pressure measurement processing.

The present embodiment differs from the first embodiment in the contents of the cognitive function test, and thus processing for cognitive function tests and check processing for answers to the test information will be described.

Cognitive Function Test Information Provision Processing

The cognitive function tests of the present embodiment is referred to as word recognition, in which the user is prompted to memorize a plurality of words displayed, and after a predetermined amount of time or after a predetermined interference task, a plurality of words are displayed that include the previously displayed words and dummy words, and the user is prompted to answer whether the currently displayed words are the previously displayed words.

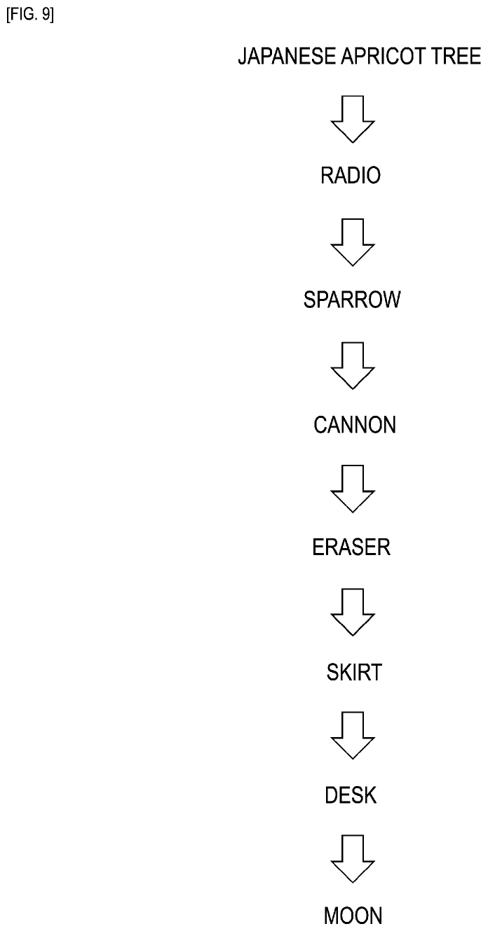

The provision processing for the cognitive function test information in the present embodiment is similar to that in FIG. 3. In this case, for example, the number n of words displayed is eight, and eight words of a Japanese apricot tree, a radio, a sparrow, a cannon, an eraser, a skirt, a desk, and the moon are sequentially displayed on the touch panel display 220 of the smartphone 20 in this order, as illustrated in FIG. 9. These words are extracted from at least three or more categories.

Answer Check Processing

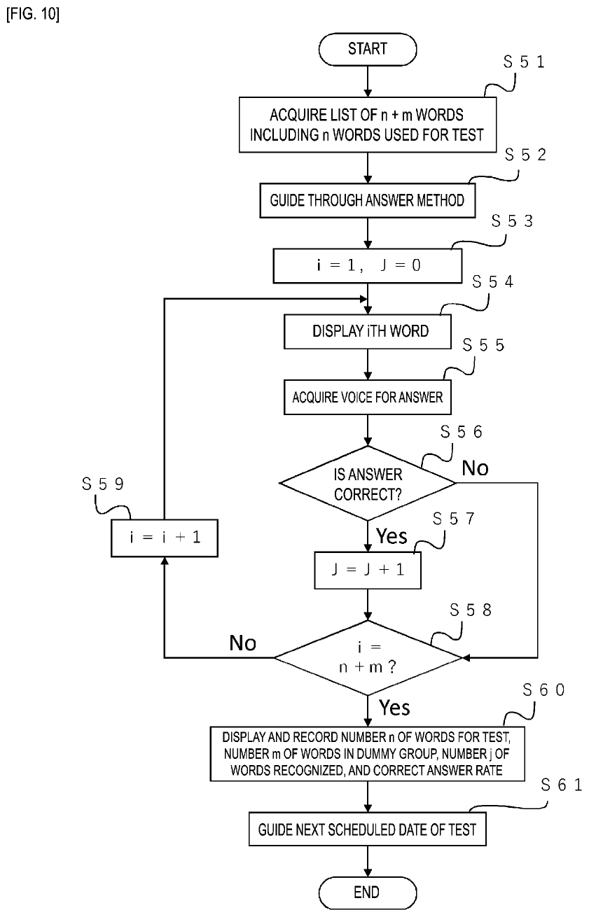

The blood pressure measurement processing is similar to that in the first embodiment, and thus the description of the blood pressure measurement processing is omitted, and the answer check processing will be described below with reference to FIG. 10.

First, n words (correct answer group) used to provide the cognitive function test information are read from the cognitive function test result recording unit 232 of the memory unit 230, and from the group of words stored in the cognitive function test information storage unit 233, m words (dummy group) not included in the correct answer group are extracted, and a word list including n+m words is acquired(step 51).

Then, a message is displayed on the touch panel display 220, the message guiding the user through the answer method such that in a case where the word displayed on the touch panel display 220 is the word displayed before the blood pressure measurement, the user orally answers “yes”, whereas in a case where the word displayed on the touch panel display 220 is not the word displayed before the blood pressure measurement, the user orally answers “no” (step S52). The message guiding the user through the answer method may be output via the voice output unit 270, and the display and the voice output may be combined.

Then, i=1 and j=0 are set (step S53). Then, the ith word is displayed on the touch panel display 220 of the smartphone 20 (step S54). In this case, as described below, any one of words included in the correct answer group or words not included in the correct answer group is displayed.

Then, the voice of the user indicating the answer “yes” or “no” is acquired via the voice input unit 260 (step S55).

The voice acquired is analyzed to determine whether the answer is correct (step S56). In other words, in step S54, in a case where the word displayed on the touch panel display 220 is included in the correct answer group and the answer acquired is “yes”, the answer is determined to be correct, and in a case where the answer acquired is “no”, the answer is determined to be incorrect. In addition, in step S54, in a case where the word displayed on the touch panel display 220 is included in the dummy group and the answer acquired is “no”, the answer is determined to be correct, and in a case where the answer acquired is “yes”, the answer is determined to be incorrect.

In step S56, in a case where the answer is determined to be correct, j=j+1 is set (step S57), and the processing proceeds to step S58. In step S56, in a case where the answer is determined not to be correct, then the processing proceeds to step S58.

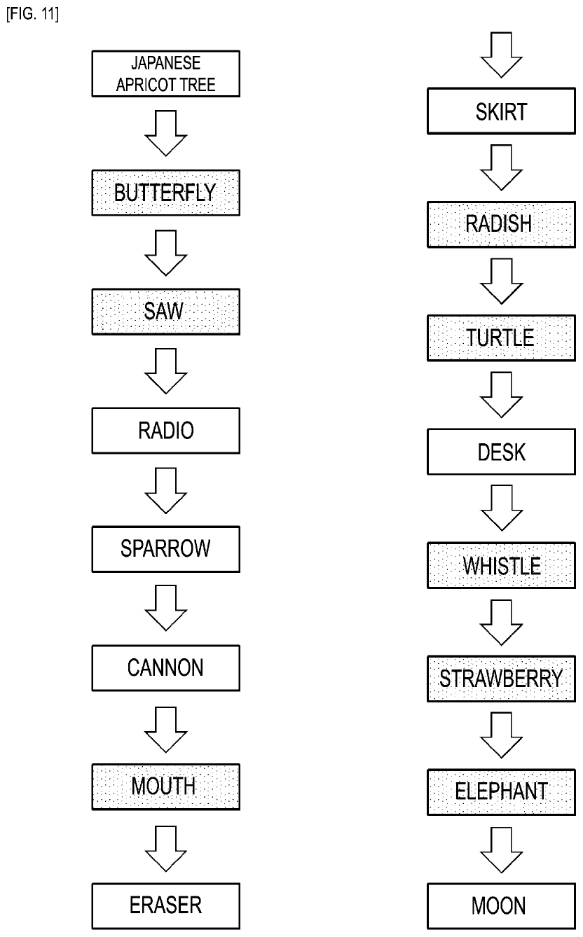

In step S58, whether i=n+m is determined. In step S58, in a case where i=n+m does not hold true, then i=i+1 is set (step S59) and the processing returns to step S54 to display the i+1th word on the touch panel display 220. In this manner, e.g., as illustrated in FIG. 11, 16 words of a Japanese apricot tree, a butterfly, a saw, a radio, a sparrow, a cannon, a mouth, an eraser, a skirt, a radish, a turtle, a desk, a whistle, a strawberry, an elephant, and the moon are displayed sequentially on the touch panel display 220 in this order. In this case, m is eight, and eight shaded words are extracted as a dummy groups. When n+m words including the correct answer group and the dummy group is displayed, the order in which the correct words are displayed is maintained as illustrated in FIG. 11, and dummy words are displayed between the correct answer words. However, the display order of the words in the correct answer group and the dummy group is not limited to this. For example, the display order of the words in the correct answer group may be changed and the dummy words may be displayed between the correct answers.

In step S58, in a case where i=n+m holds true, the number n of words used in the current cognitive function test, the number m of words added as dummies, the number j of words that can be recognized by the user, and the correct answer rate j/(n+m) are displayed on the touch panel display 220, and are recorded in the cognitive function test result recording unit 232 of the storage unit 230 (step S60).

Then, the user is guided to the next scheduled date of the cognitive function test (step S61). For example, a message is displayed on the touch panel display 220, such as “Thank you for your work, the test is over now. You have done very well. Please take the test three more times to produce final results. The next test will be conducted on Xth day.” The message may be output via the voice output unit 270.

The next scheduled date of the test is recorded in the cognitive function test result recording unit 232 of the storage unit 230, and when the blood pressure information on the day, the next cognitive function tests are prompted.

In the present embodiment as well, the number n of words displayed in the provision processing for the cognitive function test information is not limited to eight, and may be set as appropriate. In a case where the similar tests are repeated a plurality of times, the number of displayed words may be varied each time, for example, eight words on the first test, six words on the second test, and 12 words on the third test. Additionally, the number of words extracted as a dummy group may be the same number as the number of words displayed in the provision processing for the cognitive function test information, but the number of dummies can be increased or decreased to adjust the difficulty level.

Thus, according to the present embodiment, the opportunity for the measurement of the blood pressure information performed as part of routine healthcare management is utilized to conduct the cognitive function test. This enables lowering the hurdle of taking the cognitive function test, and allows early detection of dementia to be expected.

### Third Embodiment

A third embodiment of the present invention will be described below. The same reference numerals are used for components that are the same as those of the first embodiment, and detailed descriptions thereof are omitted. The present embodiment is the same as the first embodiment in the configuration of the biological information system, the overall flow from the provision of the cognitive function test information through the measurement of the blood pressure information to the check of answers to the test information, and the contents of the blood pressure measurement processing. The present embodiment differs from the first embodiment in the contents of the cognitive function test, and thus processing for cognitive function tests and check processing for answers to the test information will be described.

Cognitive Function Test Information Provision Processing

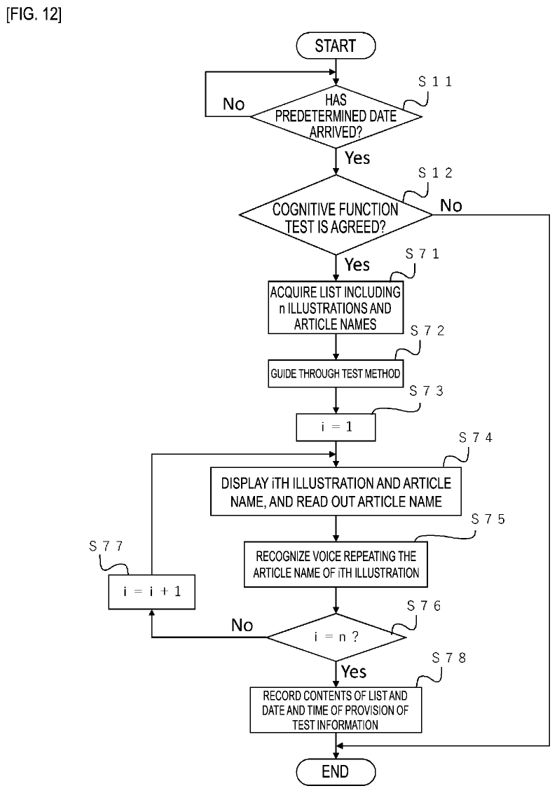

FIG. 12 is a flowchart illustrating a processing procedure for provision of the cognitive function test information. The cognitive function test of the present embodiment is referred to as word recalling using an image, in which the user is prompted to memorize the article name of displayed illustrations and to orally speak the previously displayed word after a predetermined amount of time or predetermined interference task.

The processing in step S11 and step S12 is similar to that in the first embodiment, and thus the descriptions thereof are omitted. Then, a list of n illustrations and the article names of the illustrations stored in the cognitive function test information storage unit 233 of the storage unit 230 is acquired (step S71). The cognitive function test information storage unit 233 may store the list, or may extract n sets of illustrations and article names from a group including a plurality of sets of illustrations and article names and form the sets into the list.

Then, the cognitive function test method to be performed is displayed on the touch panel display 220 of the smartphone 20 to guide through the method (step S72). In this case, a message guiding through the method of the cognitive function test is displayed, such as “Now, n illustrations are displayed one by one. The article names of the displayed illustrations are read out, and thus please repeat and memorize the article names.” The message may also be output by the voice output unit 270 of the smartphone 20.

Then, i=1 is set (step S73).

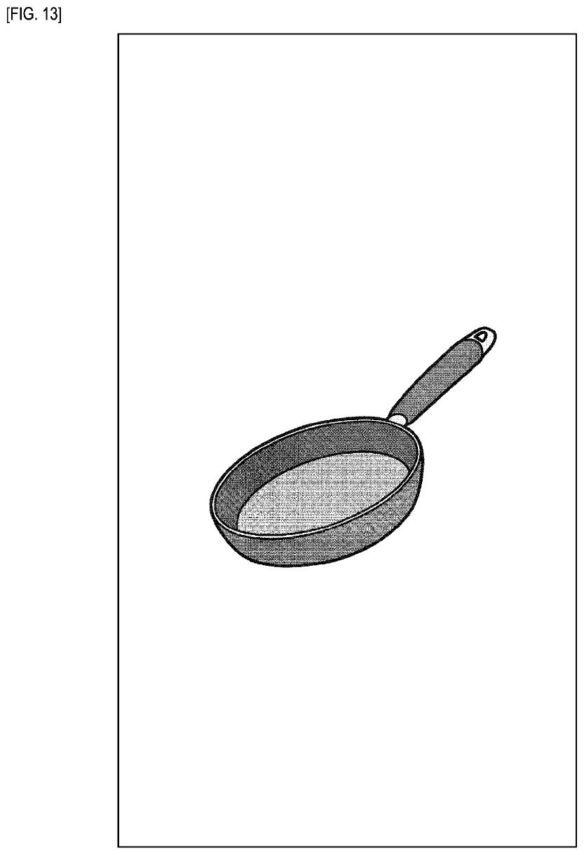

Then, as illustrated in FIG. 13, the first illustration of the list acquired in step S71 is displayed on the touch panel display 220, and the voice of reading out the article name of the illustration is output from the voice output unit 270 (step S74). In this case, an illustration of a frying pan is displayed on the touch panel display 220, and the voice “flying pan”, corresponding to an article name, is output from the voice output unit 270. At this time, a message may similarly be displayed that prompts the user to repeat the article name.

Then, from the voice acquired via the voice input unit 260 of the smartphone 20, the voice of the user repeating the article name of the ith illustration is recognized (step S75). In a case where the voice of the user reading out the ith word fails to be recognized within a predetermined amount of time, then a message prompting the user to read out the displayed word may be displayed on the touch panel display 220 or output via the voice output unit 270. Additionally, in a case where the voice of the user reading out the ith word fails to be recognized within the predetermined amount of time, then the processing may proceed to step S76.

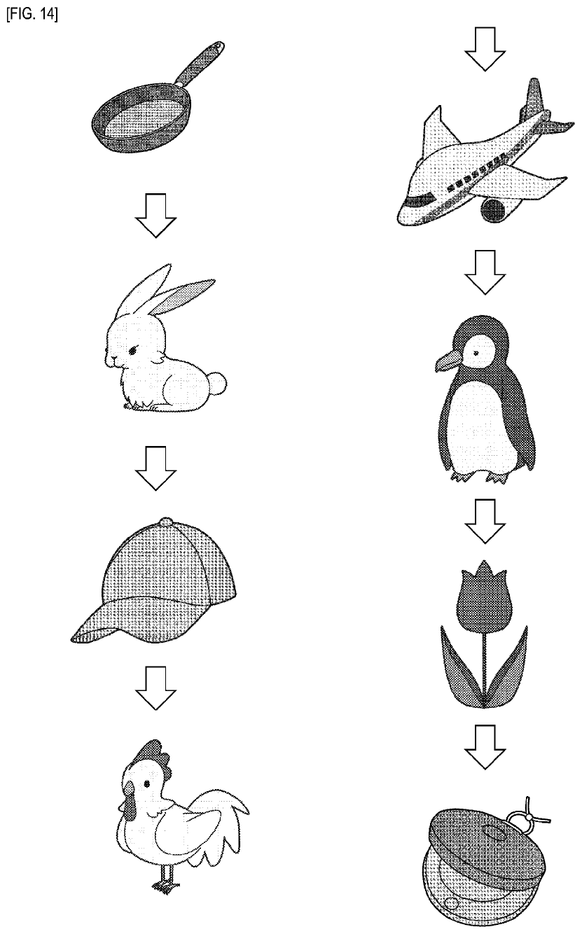

Once the voice of the user repeating the article name of the ith illustration is recognized, then whether i=n is determined (step S76). In step S76, in a case where i=n does not hold true, i=i+1 is set (step S77) and the processing returns to step S74 to repeat the processing from step S74 onward for the next illustration and the article name in the list acquired in step S71. In this way, as illustrated in FIG. 14, different illustrations are displayed sequentially on the touch panel display 220 of the smartphone 20. In this case, the list includes eight illustrations of a frying pan, a rabbit, a cap, a chicken, an airplane, a penguin, a tulip, and a castanet. The illustrations displayed at this time are all belonging to different categories. The number n of illustrations displayed is not limited to eight, and may be set as appropriate. In a case where similar tests are repeated a plurality of times, the number of displayed words may be increased each time, for example, eight words on the first test and the 12 words on the second test.

In step S76, in a case where i=n holds true, the contents of the list acquired in step S71 and the date and time of provision of test information are stored in the cognitive function test result recording unit 232 of the storage unit 230 (step S78). In this case, the date and time of provision of the test information is, for example, the date and time of recognition of the voice of reading out the nth word.

Then, the provision processing for the cognitive function test information is ended.

Answer Check Processing

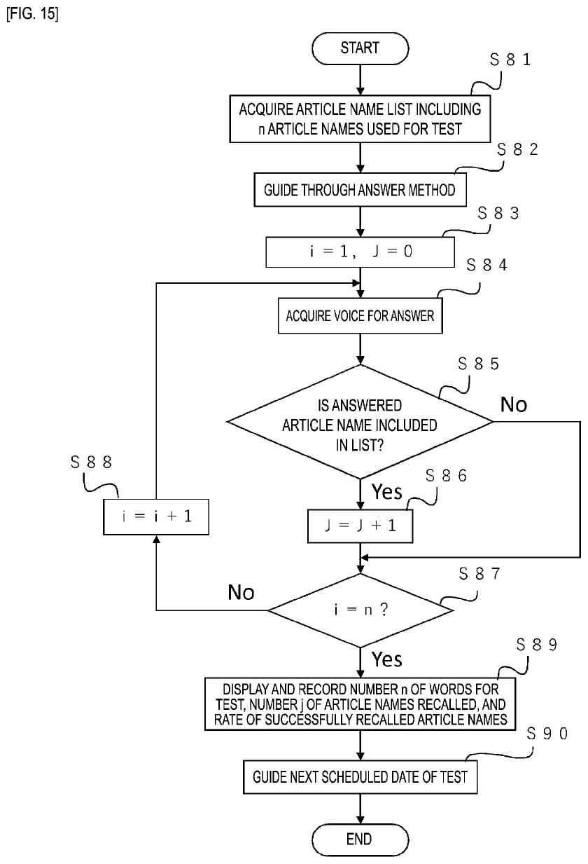

The blood pressure measurement processing is similar to that in the first embodiment, and thus the description of the blood pressure measurement processing is omitted, and the answer check processing will be described below with reference to FIG. 15.

First, an article name list including n article names used to provide the cognitive function test information is read and acquired from the cognitive function test result recording unit 232 of the storage unit 230 (step 81).

Then, a message prompting the user to orally answer memorized article names is displayed on the touch panel display 220 (step S82). For example, a message such as “Please speak about remembered article names” is displayed on the touch panel display 220. The message prompting the user to orally answer the memorized article names may be output via the voice output unit 270, and the display and the voice output may be combined.

Then, i=1 and j=0 are set (step S83). Then, the voice of the user for the ith word is acquired via the voice input unit 260 (step S84).

The voice acquired is analyzed to determine whether the article name is included in the article name list acquired in step S81 (step S85). In step S85, in a case where the article name is determined to be included in the article name list, j=j+1 is set (step S86) and the processing proceeds to step S87.

In step S85, in a case where the article name is determined not to be included in the article name list, then the processing proceeds to step S87.

In step S87, whether i=n is determined. In step S87, in a case where i=n does not hold true, then i=i+1 is set (step S88) and the processing returns to step S84 to wait to acquire the voice for the i+1th word.

In step S87, in a case where i=n holds true, the number n of article names used in the current cognitive function test, the number j of article names successfully recalled by the user, and the correct answer rate j/n are displayed on the touch panel display 220, and are recorded in the cognitive function test result recording unit 232 of the storage unit 230 (step S89).

Then, the user is guided to the next scheduled date of the cognitive function test (step S90). For example, a message is displayed on the touch panel display 220, such as “Thank you for your work, the test is over now. You have done very well. Please take the test three more times to produce final results. The next test will be conducted on Xth day.” The message may be output via the voice output unit 270.

The next scheduled date of the test is recorded in the cognitive function test result recording unit 232 of the storage unit 230, and when the blood pressure information on the day, the next cognitive function tests are prompted. Thus, accuracy of evaluation is increased by conducting the cognitive function test a plurality of times.

Thus, according to the present embodiment, the opportunity for the measurement of the blood pressure information performed as part of routine healthcare management is utilized to conduct the cognitive function test. This enables lowering the hurdle of taking the cognitive function test, and allows early detection of dementia to be expected.

### Fourth Embodiment

In the present embodiment, the biological information has been acquired by receiving transfer of biological information already measured by the biological information measurement instrument. In other words, the biological information acquisition processing is implemented as a transferred biological information reception processing. The same reference numerals are used for components and processing that are common as those of the first embodiment, and the detailed descriptions thereof are omitted. The biological information includes, for example, but is not limited to, body weight, body composition, exercise amount, or body temperature, in addition to blood pressure.

The schematic configuration of the biological information acquisition system 1 used as a biological information acquisition device is same as that in the first embodiment illustrated in FIG. 1. However, in the present embodiment, the blood pressure monitor 10 stores, in the measurement result recording unit 170 of the storage unit 160, the blood pressure information as the biological information that has already been measured. Then, when the blood pressure monitor 10 transfers, to the smartphone 20, measurement results stored in the measurement result recording unit 170, the smartphone 20 acquires the blood pressure information recorded in the measurement result recording unit 170 of the blood pressure monitor 10. In this regard, the blood pressure monitor 10 will be described as an example, but a plurality of biological information measurement instruments including a body weight meter, a body composition analyzer, an exercise meter, a clinical thermometer, and the like, which are communicably connected to the smartphone 20, may be configured to transfer biological information stored in the biological information measurement instruments. Additionally, a device that is communicably connected to the smartphone 20 and transfers biological information may be a biological information measurement instrument having a biological information measurement function, or a device having no biological information measurement function but storing biological information measured in another biological information measurement instrument. Furthermore, the smartphone 20 may receive, from a device communicably connected to the smartphone 20, transfer of related information as information having some association with biological information including environmental conditions such as air temperature and atmospheric pressure stored in a predetermined region of the storage unit 160. In this regard, a biological information measurement instrument having a biological information measurement function, and a device that has no biological information measurement function but that stores biological information and related information measured in another biological information measurement instrument are collectively referred as biological information holding device.

The processing procedure for the biological information acquisition method including the cognitive function test processing, the biological information acquisition processing, and the answer check processing is similar to that in the first embodiment illustrated in FIG. 2.

Cognitive Function Test Information Provision Processing

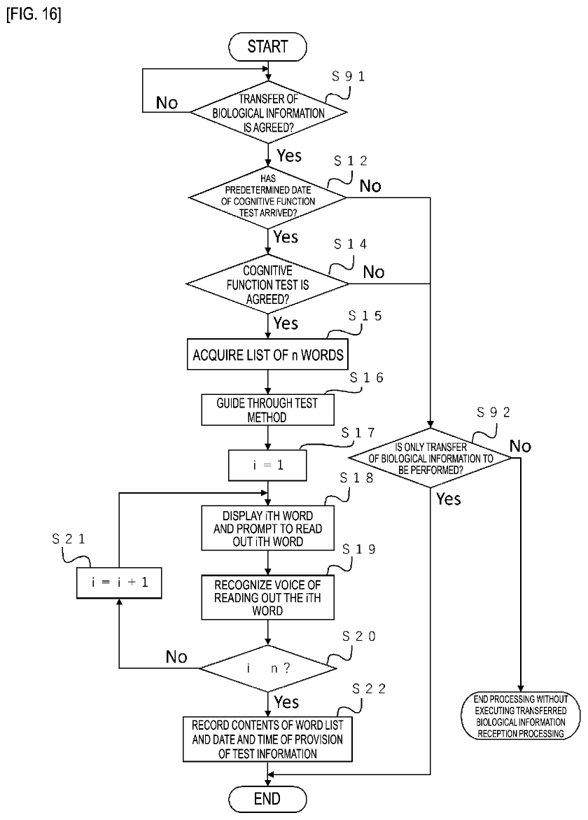

FIG. 16 illustrates the cognitive function test information provision processing. Only differences from the cognitive function test information provision processing of the first embodiment illustrated in FIG. 3 will be described.

In the present embodiment, a message “Do you like to receive transfer of biological information?” is displayed on the touch panel display 220 of the smartphone 20, or the voice for the message is output from the voice output unit 270, to ask the user to agree with the transfer of the biological information and the like (step S91).

In a case where a plurality of biological information holding devices are communicably connected to the smartphone 20, the user may be asked to agree by selecting the biological information holding devices, from which transfer of the biological information is to be received, from a list of a plurality of biological information holding devices including a blood pressure monitor 10 displayed on the touch panel display 220 of the smartphone 20.

The processing in step S12 is similar to that in the first embodiment. In step S12, in a case where the cognitive function test date is determined not to have arrived, the user is prompted by the touch panel display 220 or the voice output unit 270 to determine whether only transfer the biological information or the like is to be performed (step S92).

In step S92, in a case where an input indicating that only the transfer of the biological information is to be performed is received from the user via the touch panel display 220 or via the voice input unit 260, the cognitive function test information provision processing is ended, and the transferred biological information reception processing described below is performed. In this case, in a case where the transfer biological information reception processing ends, the processing is ended without performing the answer check processing. In step S92, in a case where an input indicating that only the transfer of the biological information is not to be performed is received from the user via the touch panel display 220 or via the voice input unit 260, the processing is ended without performing both the transferred biological information reception processing and the answer check processing.

Transferred Biological Information Reception Processing

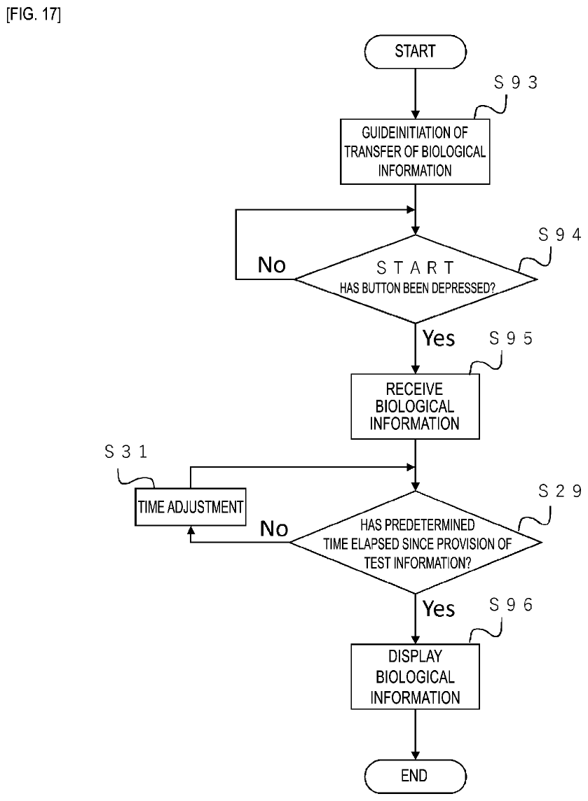

FIG. 17 is a flowchart illustrating a processing procedure for reception of the transferred biological information. The transferred biological information reception processing described below is implemented by the control unit 240 of the smartphone 20 executing a program stored in the storage unit 230 and cooperating with the blood pressure monitor 10. At this time, in the blood pressure monitor 10, the control unit 150 similarly executes a program stored in the storage unit 160. A biological information acquisition unit includes the touch panel display 220, the storage unit 230, the clock unit 250, the voice input unit 260 and the voice output unit 270, the communication unit 210, and the control 240 that are belonging to the smartphone 20, in which the control unit 240 cooperates with the units 220, 230, 250, 260, 270, and 210. The biological information acquisition unit further includes at least the storage unit 160, the communication unit 130, and the control unit 150 that are belonging to the blood pressure monitor 10, in which the control unit 150 cooperates with the units 160 and 130.

First, the initiation of the transfer of the biological information is guided to the user (step S93). At this time, the “START” button is displayed on the touch panel display 220 of the smartphone 20, and a message “Press the START” button or say “start” to initiate “transfer.”, which guides the initiation of the transfer, is displayed on the touch panel display 220 or output from the voice output unit 270.

The smartphone 20 waits for the user to depress the “START” button displayed on the touch panel display 220 of the smartphone 20 or waits for the user to input the voice “start” via the voice input unit 260 (step 94). When depression of the “START” button or the voice “start” is detected, the blood pressure information stored in the measurement result recording unit 170 of the blood pressure monitor 10 is received (step S95). In a case where a plurality of biological information holding devices from which the transfer of the biological information is to be received are selected, the biological information is received sequentially from each of the biological information holding devices.

The processing in steps S29 and S31 is similar to that in the first embodiment illustrated in FIG. 6. In step S29, in a case where the determination is made that a predetermined amount of time has elapsed since the date and time of provision of the cognitive function test information, the biological information received in step S95 is displayed on the touch panel display 220 of the smartphone 20 (step S96), and the processing is ended. In step S96, the received biological information may be output via the voice output unit 270.

Thus, according to the present embodiment, the opportunity for the transfer of the biological information measured as part of routine healthcare management is utilized to conduct the cognitive function test. This enables lowering the hurdle of taking the cognitive function test, and allows early detection of dementia to be expected.

### MODIFIED EXAMPLES

The processing in step S26 and step S28 in the blood pressure information measurement processing may be performed in another procedure in the blood pressure information measurement processing.

The contents of the cognitive function test are not limited to the contents described in the first embodiment, the second embodiment, and the third embodiment, and any other cognitive function test may be employed, and the cognitive function tests described in the first embodiment, the second embodiment, and the third embodiment may be combined.

Additionally, by changing test information such as the numbers of words and illustrations provided as the cognitive function test information, the difficulty level, and the like, the amount of time required after the end of the cognitive function test information provision processing until the initiation of the answer check processing can be extended or shortened. Thus, the test information may be changed such that the above-described amount of time is equal to the amount of time required for the blood pressure information measurement processing.

In the first embodiment, the biological information measurement system has been described that includes, as the biological information measurement instrument, the blood pressure monitor that measures the blood pressure information. The present invention can also be applied to a biological information measurement system that includes, as the biological information measurement instrument, a body weight meter measuring the body weight as the biological measurement information, a body composition analyzer measuring the body composition such as the body fat percentage, an exercise meter measuring the exercise amount such as the number of steps or calorie consumption, and a clinical thermometer measuring the body temperature.

In addition, in the first embodiment, the blood pressure monitor 10 is configured to measure blood pressure information alone, but in FIG. 1, the input unit 140 and the display unit 120 may be omitted, and input such as operation information and the display of various information can be configured to be performed on the smartphone 20 side. Additionally, in FIG. 1, the measurement result recording unit 170 may be omitted, and the measurement results may be configured to be recorded on the smartphone 20 side.

The biological information measurement system according to the first embodiment is configured to include a smartphone 20 as a terminal, but the terminal is not limited thereto, and may be a stationary information terminal such as a tablet terminal, a laptop computer, or the like.

In addition, in the first to third embodiments, as illustrated in FIG. 2, the blood pressure information is measured between the provision of the cognitive function test information and the check of answers from the user to the cognitive function test information. In a case where each of the processing are performed in this order, for a test in which both an answer immediately after memorization (the first answer) and an answer some time after the memorization (the second answer) are checked, the provision of the cognitive function test information and the check of the first answer from the user to the cognitive function test information may be performed before the measurement of the blood pressure information, and the check of the second answer from the user to the cognitive function test information may be performed after the measurement of the blood pressure information. In a case where cognitive function tests are conducted that vary in the time to elapse which is required before an answer is given and vary in the interference task, for a test with a shorter time to elapse, the first answer check is performed before the measurement of the blood pressure information, and for a test with a longer time to elapse, the second answer check may be performed after the measurement of the blood pressure information. In addition, for the same cognitive function test that varies in the time to elapse which is required before an answer is given and varies in the interference task, the answer check may be performed before and after the measurement of the blood pressure information.

Additionally, in the blood pressure information measurement processing illustrated in FIG. 6, the initiation of the blood pressure measurement is guided after the end of the cognitive function test information provision processing. In the biological information measurement processing including, as an example, the blood pressure information measurement processing as described above, an exercise task such as stepping or step-ups exercise may be assigned to the user before the biological information is measured, and after the task, the biological information may be measured. This allows the exercise task to be incorporated into the measurement of the biological information as a method for time adjustment. In this case, time adjustment can be made by displaying the timing of stepping and the number of steps in the step-ups exercise on the touch panel display 220 of the smartphone 20 or outputting the timing of stepping and the number of steps from the voice output unit 270.

The measurement of the biological information described in the first to third embodiments may be combined with the transfer of the biological information described in the fourth embodiment. In this case, the measurement of the blood pressure information by the blood pressure monitor 10 can be combined with the transfer of the blood pressure information or the measurement of the blood pressure information by the blood pressure monitor 10 can be combined with the transfer of the biological information from another biological information measurement instrument.

### REFERENCE SIGNS LIST

1 Biological information measurement system
10 Blood pressure monitor
20 Smartphone
110 Sensor unit
130 Communication unit
150 Control unit
160 Storage unit
210 Communication unit
220 Touch panel display
230 Storage unit
240 Control unit
250 Clock unit
260 Voice input unit
270 Voice output unit

1. A biological information acquisition device including a cognitive function test function, the biological information acquisition device comprising:
a test information provision unit configured to perform test information provision processing that provides a user with test information for testing cognitive function;
biological information acquisition unit configured to perform biological information acquisition processing that acquires biological information of the user; and
answer check unit configured to perform answer check processing in which the biological information acquisition device checks answers from the user to the test information, wherein
the test information provision processing, the biological information acquisition processing, and the answer check processing are performed in this order.

2. The biological information acquisition device according to claim 1, wherein an amount of time for the biological information acquisition processing is matched with an amount of time that needs to elapse after end of the test information provision processing until initiation of the answer check processing.

3. The biological information acquisition device according to claim 2, wherein contents of the test information are changed such that an amount of time that needs to elapse after the end of the test information provision processing until the initiation of the answer check processing is equal to an amount of time needed for the biological information acquisition processing.

4. The biological information acquisition device according to claim 2, wherein a length of time for performing the biological information acquisition processing is determined such that the biological information acquisition processing is performed during an amount of time that needs to elapse after the end of the test information provision processing until the initiation of the answer check processing.

5. The biological information acquisition device according to claim 1, wherein the biological information acquisition processing is processing that acquires the biological information by measurement.

6. The biological information acquisition device according to claim 1, wherein the biological information acquisition processing is acquisition processing that receives transfer of the biological information measured by a biological information measurement instrument.

7. A biological information acquisition method executed in a biological information acquisition device including a cognitive function test function, the biological information acquisition method comprising:
(a) providing a user with test information for testing cognitive function;
(b) acquiring biological information of the user; and
(c) the biological information acquisition device checking answers from the user to the test information, wherein
(a), (b) and (c) are performed in this order.
